(19) United States
(12) Patent Application Publication
(10) Pub. No.: US 2024/0203605 A1
(43) Pub. Date: Jun. 20, 2024

(54) CONVOLUTIONAL NEURAL NETWORK FOR SUBVISIBLE PARTICULATE CLASSIFICATION OF BIOPHARMACEUTICALS

(71) Applicant: Merck Sharp & Dohme LLC, Rahway, NJ (US)

(72) Inventors: Shubing Wang, Westfield, NJ (US); Andy I-Hsuen Liaw, Plainsboro, NJ (US); Yue-Ming Chen, New Providence, NJ (US); Yongchao Su, Hillsborough, NJ (US); Daniel Skomski, East Brunswick, NJ (US)

(21) Appl. No.: 18/390,291

(22) Filed: Dec. 20, 2023

Related U.S. Application Data
(60) Provisional application No. 63/433,965, filed on Dec. 20, 2022.

(51) Int. Cl. G16H 70/40; G06N 3/0464; G06T 7/00

(52) U.S. Cl. CPC G16H 70/40; G06N 3/0464; G06T 7/0012

(57) ABSTRACT

An analysis system implements a particulate classification model trained as a convolutional neural network to assess sterile formulation quality. The system obtains micro flow imaging (MFI) data for a sterile formulation drug product, wherein the MFI data includes an image depicting a plurality of sub-visible particulates in the sterile formulation drug product. The system detects a plurality of features in the image, wherein each feature is bounded by a bounding box. The system performs preprocessing to determine and remove one or more of features to be artifacts based on characteristics of the features, wherein the remaining features are determined to be sub-visible particulates. The system applies a particulate classification model to each sub-visible particulate to determine a particulate classification label. The system determines a quality metric for the sterile formulation drug product based on a count of sub-visible particulates in each particulate classification label.

### CROSS-REFERENCE TO RELATED APPLICATIONS

The present application claims the benefit of and priority to U.S. Provisional Application No. 63/433,965 filed on Dec. 20, 2022, which is incorporated by reference in its entirety.

### BACKGROUND

### 1. Technical Field

The subject matter described relates generally to particulate classification and, in particular, to convolutional neural networks that provide accurate and automated classification of subvisible particles.

### 2. Background Information

Globally a substantial need exists for the development of new pharmaceutical treatments to address a broad spectrum of human diseases. Biologics are commonly formulated in the form of sterile liquids which can be administrated by various means, including intravenous infusion, subcutaneous delivery, and others. In recent years, high-concentration subcutaneous injection from a prefilled syringe (PFS) has attracted increasing attention for its convenience to health care providers and patients. Sterile products require stringent control of critical quality attributes (CQAs) in manufacturing and storage across the product's lifecycle.

One mechanism of failure in such formulations involves particulation occurring at various size scales. Subvisible particulation in biopharmaceuticals (e.g., involving particles ranging in size from 2 to 100 μm) is a high-complexity space, with strict regulatory implications for sterile formulation drug products and potential adverse product quality impact. Micro flow imaging (MFI) is a useful tool for detecting subvisible particles and measuring morphological features across statistically-relevant populations, including for monoclonal antibody therapies, vaccines, and small molecules. MFI is leveraged in the pharmaceutical industry as an orthogonal extended characterization method. With an increased representation of high-complexity combination products in the biopharmaceutical development pipeline, such as for subcutaneous administration, MFI is heavily utilized as a powerful technique to investigate the nature of the particle species for monitoring protein stability.

MFI provides quantifiable morphological parameters to study both the size and type of subvisible particles. However, limitations in existing approaches result can result in inaccurate particle classification. While MFI provides enormously rich information content, the particle morphologies, colors, and textures from MFI are difficult to accurately describe mathematically because of their enormous complexity and diversity, which can hinder a more in-depth quantitative understanding. Industrial procedures have been developed based on morphological distinction via simple image-based filters like particle aspect ratio and particle mean intensity, but these methods have limitations in their ability to classify subvisible particles accurately and efficiently.

### SUMMARY

The above and other problems can be addressed using a particle classifier that applies a particulate classification model to classify particulates. Using this approach, features such as air bubbles, silicone oil droplets, protein aggregates, and instrument artifacts may be accurately identified and accounted for during analysis. This analysis may be performed automatically or semi-automatically and reduce false positive rates (and other errors) relative to conventional approaches. In one or more embodiments, the particulate classification model is optimized for classification of small-sized particulate images. Accordingly, the particulate classification model may be trained to optimize classification performance while minimizing a size of the particulate classification model, i.e., to minimize computations. This approach may also increase analysis speed, allowing for a greater efficiency in particle classification.

In one embodiment, an analysis system implements a particulate classification model trained as a convolutional neural network to assess sterile formulation quality. The system obtains imaging data (e.g., micro flow imaging data) for a sterile formulation drug product, wherein the imaging data includes an image depicting a plurality of sub-visible particulates in the sterile formulation drug product. The system detects a plurality of features in the image, wherein each feature is bounded by a bounding box. The system performs preprocessing to determine and remove one or more of features to be artifacts based on characteristics of the features, wherein the remaining features are determined to be sub-visible particulates. The system applies a particulate classification model to each sub-visible particulate to determine a particulate classification label. The system determines a quality metric for the sterile formulation drug product based on a count of sub-visible particulates in each particulate classification label.

### BRIEF DESCRIPTION OF THE DRAWINGS

FIG. 1 illustrates one embodiment of a networked computing environment generating and analyzing MFI data, according to one or more embodiments.

FIG. 2 illustrates a block diagram representing an architecture of the analysis system of FIG. 1, according to one or more embodiments.

FIG. 3A illustrates the method 300 of particulate classification model training, according to one or more embodiments.

FIG. 3B illustrates the method 340 of particulate classification model deployment, according to one or more embodiments.

FIG. 4 is a block diagram illustrating an example of a computer suitable for use in the networked computing environment of FIG. 1, according to one embodiment.

FIG. 5 illustrates a micro flow image, according to an example implementation.

FIG. 6 illustrates example particulates classified into each classification label, according to an example implementation.

FIG. 7 illustrates example results comparing the particulate classification methodology with a conventional particulate screening methodology, according to an example implementation.

### DETAILED DESCRIPTION

### Overview

An analysis system implements a particulate classification model to classify sub-visible particulates in imaging data for a sterile formulation, e.g., micro flow imaging (MFI) data captured by an MFI system. The particulate classification model may be trained as a convolutional neural network to accurately and precisely classify the sub-visible particulates. Prior to classification, the analysis system may perform preprocessing steps to remove artifacts from the MFI data, e.g., including stripe-type artifacts and/or dirty-lens-type artifacts. Once classified, the analysis system can generate a quality metric for the MFI data to assess the quality of the sterile formulation. The algorithms described throughout can also be readily applied to other types of imaging data of sub-visible particulates.

The particulate classification model has various applications including improving drug manufacturing pipelines by functioning as a quality control tool for drug formulation batches. This model can assess consistency between different manufacturing batches, identify potential quality degradation, and compare different manufacturing approaches. Additionally, it can assess potential changes in the quality of drug formulations under various conditions, contributing to a better understanding of the drug's shelf life. Another application includes characterizing different drug formulations, for example, assessing the quality of different types of monoclonal antibodies in drug formulations to determine the most effective option for production and marketing, thus serving as a versatile tool for efficiency and optimization in the pharmaceutical industry.

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### System Environment

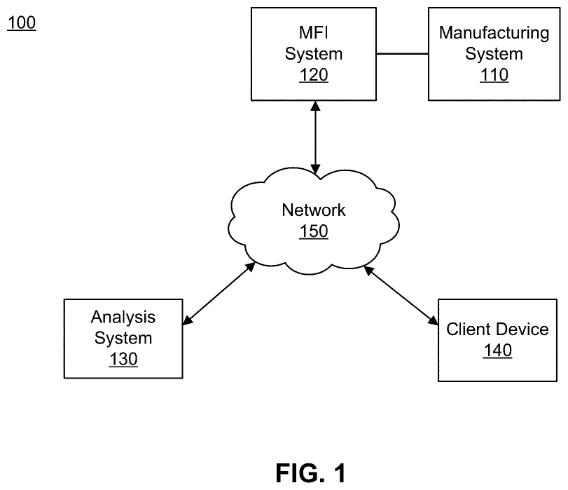

FIG. 1 illustrates one embodiment of a networked computing environment 100 generating and analyzing MFI data. In the embodiment shown, the networked computing environment includes a manufacturing system 110, an MFI system 120, an analysis system 130, and a client device 140, all connected via a network 150. In other embodiments, the networked computing environment 100 includes different or additional elements. In addition, the functions may be distributed among the elements in a different manner than described. Furthermore, in another embodiment, the described functionality may be performed by a single computing device that is not connected to a network.

The manufacturing system 110 generates sterile formulation drug products. The manufacturing process may entail: formulation, compound mixing, filling, sterilization, sealing and labeling, inspection, packaging, or some combination thereof. Sterile drug products are manufactured through a carefully controlled and aseptic process. Formulation includes selecting the appropriate pharmaceutical ingredients (APIs) and any necessary excipients. Formulation may include testing and research to determine optimal conditions for drug efficacy and stability. Compound Mixing entails mixing of the components, e.g., often taking placing place in a sterile, controlled environment to reduce possibilities of contamination. Filling entails filling the mixed compound into chosen delivery containers using an aseptic filling machine. These containers are often glass vials, ampoules, pre-filled syringes, or infusion bags. Sterilization entails ensuring the removal of any contaminants. Sterilization may be accomplished via steam sterilization, radiation, filtration, or some combination thereof. Sealing and labeling entails sealing the sterile formulation and preparing the product with the appropriate labels for shipment. Inspection entails a post-production review of all products to validate sterility, safety and efficacy before being packaged for distribution. Packaging entails storing the products for distribution. The manufacturing system 110 may include one or more devices associated with various steps of the manufacturing process. Throughout the manufacturing process, one or more of the devices may be fully automated or may involve human operation.

The MFI system 120 generates MFI data describing properties of particles in a sample. In one embodiment, the MFI system includes imaging hardware (e.g., an MFI5200 system) and control software (e.g., MFI View System Software), which may be run on the imaging hardware itself or a connected computer. The MFI data includes micro-flow open images of the samples. The control software detects particles having a size greater than a threshold (e.g., 1 μm) and supplements the images with bounding boxes around detected particles. The control software may also calculate morphological features of the particles, which may be saved as metadata associated with the images. The MFI system 120 may generate the MFI data as any combination of the above attributes. For example, the MFI data may include just the imaging data without any imaging analysis. In other examples, the MFI data may include the imaging data with some or all of the imaging analysis performed by the control software.

The MFI system 120 operates in conjunction with the manufacturing system 110. The manufacturing system 110 may provide samples of the sterile formulation to the MFI system 120 for imaging and subsequent analysis. In some embodiments, the MFI system 120 may be a component of the manufacturing system 110. In other embodiments, the MFI system 120 may communicate with the manufacturing system 110 via the network 150.

The analysis system 130 performs one or more analyses on the MFI data captured by the MFI system 120. Example analyses include preprocessing MFI data for a sample, particulate classification of sub-visible particulates, generating a quality metric for a sample based on the classification labels of the sub-visible particulates, other analyses related to the sub-visible particulates identified in the MFI data, other analyses related to the MFI data. In one or more embodiments, the analysis system 130 preprocesses the MFI data and applies a particulate classification model (e.g., a CNN) to classify sub-visible particulates identified from the MFI data. The analysis system 130 may label images in the MFI data with labels indicating the determined classification of the sub-visible particulates. The analysis system 130 may further generate a quality metric based on the classification labels of the sub-visible particulates.

The client device 140 is a computing device with which a user may interact with the other elements of the networked computing environment (e.g., a terminal, laptop, tablet, smartphone, or any other suitable computing device). In one embodiment, a may use the client device 140 to modify or otherwise control the manufacturing process implemented by the manufacturing system 110, initiate image capture by the MFI system 120, view the results generated by the analysis system 130, or some combination thereof. Similarly, the client device 140 may be used to configure the manufacturing system 110, the MFI system 120, the analysis system 130, or some combination thereof (e.g., to provide an updated classifier or set parameters for imaging).

The network 150 provides the communication channels via which the other elements of the networked computing environment 100 communicate. The network 150 can include any combination of local area and wide area networks, using wired or wireless communication systems. In one embodiment, the network 150 uses standard communications technologies and protocols. For example, the network 150 can include communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 150 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 150 may be represented using any suitable format, such as hypertext markup language (HTML) or extensible markup language (XML). In some embodiments, some or all of the communication links of the network 150 may be encrypted using any suitable technique or techniques.

### Analysis System Architecture

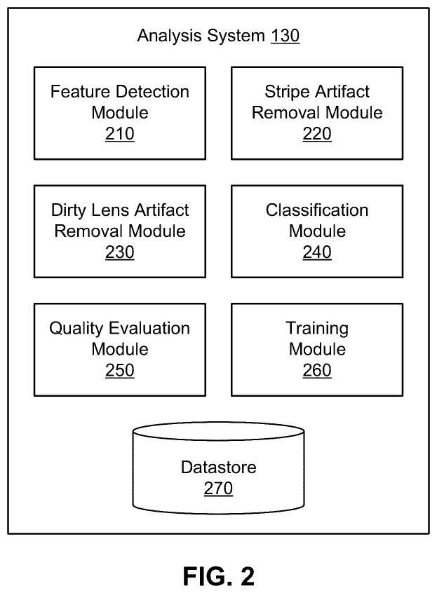

FIG. 2 illustrates a block diagram representing an architecture of the analysis system 130 of FIG. 1, according to one or more embodiments. In the embodiments shown, the analysis system 130 includes a feature detection module 210, a stripe artifact removal module 220, a dirty lens artifact removal module 230, a classification module 240, a quality evaluation module 250, a training module 260, and a datastore 270. In other embodiments, the analysis system 130 includes different or additional elements. In addition, the functions may be distributed among the elements in a different manner than described.

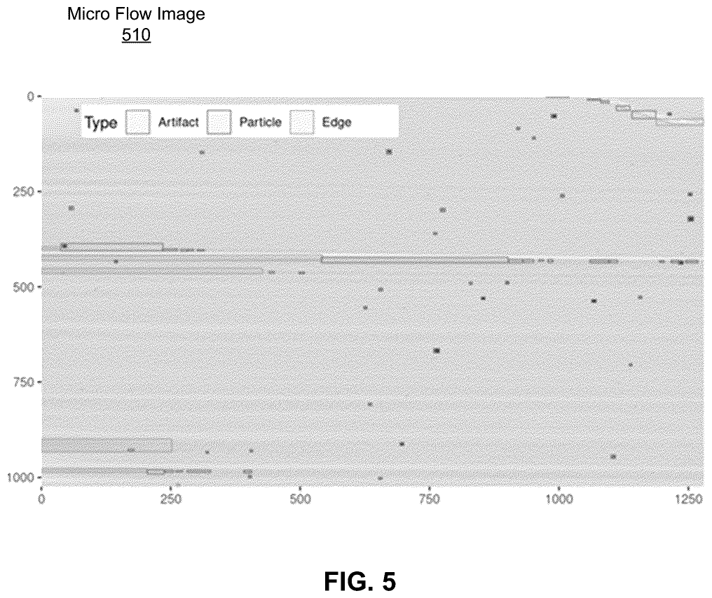

The feature detection module 210 identifies features from the MFI data. In some embodiments, the MFI data comprises just the imaging data captured by the MFI system 120. In such embodiments, the feature detection module 210 may identify the features from the imaging data. The feature detection module 210 may identify the features based on a contrast with a background signal. The background signal may be determined as the signal level of the majority of pixels in the MFI data. For example, in FIG. 5, the feature detection module 210 may identify the signal value (e.g., values for light grey) as the background signal. The feature detection module 210 may then identify features having above a threshold contrast with the background signal. The feature detection module 210 may further aggregate adjacent and/or contiguous features deemed to be a single feature. For example, if two features are within a threshold distance (e.g., less than 10, 9, 8, 7, 6, 5, 4, 3, or 2 pixels), then the feature detection module 210 may aggregate the two features into one single feature. The feature detection module 210 may further determine a bounding box for each feature. The bounding box for a feature may be a rectilinear polygon crop of the MFI data centered around the feature.

The stripe artifact removal module 220 removes stripe-type artifacts from the MFI data. Stripe-type artifacts are features in the MFI data with a threshold aspect ratio (e.g., less than 0.3, 0.2, 0.1, 0.05, etc.), appearing as long horizontal stripes in images generated by the MFI system 120. As shown in FIG. 5, the stripe artifact removal module 220 may tag the stripe-type artifacts accordingly to exclude such artifacts from biasing the particulate classification.

The dirty lens artifact removal module 230 removes dirty-lens-type artifacts from the MFI data. Dirty-lens-type artifacts appear in the same approximate coordinates in images over many frames leading to multi-counting. The dirty lens artifact removal module 230 may differentiate between dirty-lens-type artifacts from stuck particles that persist in numerous images in the MFI data. Both types of features persist in numerous images in the MFI data. The dirty-lens-type artifacts may be differentiated for having a sufficiently different shape than the particulates and/or being of a sufficiently different size than the particulates.

In some embodiments, the stripe artifact removal module 220 and the dirty lens artifact removal module 230 each perform part of the data preprocessing. In other embodiments, different or additional modules may perform preprocessing operations on the MFI images. Features that are not deemed artifacts are deemed to be sub-visible particulates. In some embodiments, additional preprocessing may further assess whether particulates are positioned on the edge of the image. If so, the particulates are deemed edge artifacts and excluded from the classification process. This is advantageous so as to not skew the classification results that would otherwise include such eclipsed particulates.

The classification module 240 applies a particulate classification model to the sub-visible particulates to determine a particulate classification label for each particulate. In one embodiment, each particulate is assigned one of a group of possible particulate classification labels. For example, the classification module 240 may classify each image into one of the following categories: Air Bubble, Background, Air-Silicone Hybrid, Protein Aggregate, or Silicone Oil. In some embodiments, the classification module 240 may calculate a probability that each image falls into one of the possible classifications and assign the classification with the highest probability. If no classification exceeds a threshold probability (or multiple classifications exceed a threshold probability), the classification module 240 may flag the image for human review to determine the correct classification.

In one or more embodiments, the particulate classification model is a convolutional neural network (CNN). A CNN is a class of deep learning neural networks. CNNs are advantageous at learning spatial hierarchies of features from image data. Generally, a CNN comprises a combination of the following: one or more convolutional layers, one or more pooling layers, one or more fully connected layers, and an output layer. The CNN is capable of identifying different patterns in an input image to predict a classification label of the sub-visible particulates in the input image. In other embodiments, the particulate classification model is another type of machine-learning model. Example machine-learning algorithms include: a multi-layer perceptron, a recurrent neural network, an autoencoder, decision trees, etc.

The quality evaluation module 250 generates a quality metric for an MFI sample based on the particulate classification labels. The quality evaluation module 250 may determine the quality metric based on a count of the particulate classification labels. For example, the quality metric may range from [0, 100], where 100 is the highest quality value and 0 is the lowest quality value. The quality evaluation module 250 may utilize one of a plurality of functions based on a particular implementation. For example, one function may return a low quality value for a large count of sub-visible particulates with the classification label of protein aggregate, and contrarily a high quality value for a small count of sub-visible particulates with the classification label of protein aggregate. In another example, another function could also factor in particulates classified as air bubbles, silicone oil, the air-silicone hybrid, or some combination thereof. Yet another function could differentially weight the effects of each particulate classification label on the quality metric. For example, the function utilizes a weight vector that scales each count of sub-visible particulates in each classification label.

The training module 260 trains the particulate classification model. The training module 260 obtains training data for training the particulate classification model. The training data may be in the form of cropped image data of sub-visible particulates with annotated labels of each particulate classification. The sub-visible particulates may be obtained from MFI data captured by an MFI system (e.g., the MFI system 120) or another imaging device capable of sub-visible particulate imaging. Other components of the analysis system 130 may aid in obtaining the training data from the MFI data, e.g., the feature detection module 210 may identify the sub-visible particulates in the MFI data, the artifact removal modules may remove artifacts from the features identified by the feature detection module 210, etc.

In other embodiments, the training data may be unlabeled, and the particulate classification model identifies the particulate classifications. The training module 260 may identify the optimal division of particulate classifications. To do so, the training module 260 may train multiple particulate classification models with the same training data but with different numbers of classes. For example, a first model is trained to classify between two classes, a second model is trained to classify between three classes, a third model is trained to classify between four classes, and so on. The training module 260 may implement a penalization to minimize the number of classes whilst optimizing predictive accuracy (e.g., optimizing sensitivity at a given specificity).

The datastore 270 includes one or more computer-readable media that store the data used and/or generated by the other modules in the analysis systems 120. For example, the datastore 270 may include the trained models. The datastore 270 may further include the MFI data and/or derivative data thereof. For example, the datastore 270 may include the raw MFI data received from the MFI system 120, and may further include cropped images of features identified by the feature detection module 210, artifacts identified by the artifact removal modules, classification labels predicted by the classification module 240, or some combination thereof.

### Exemplary Methods

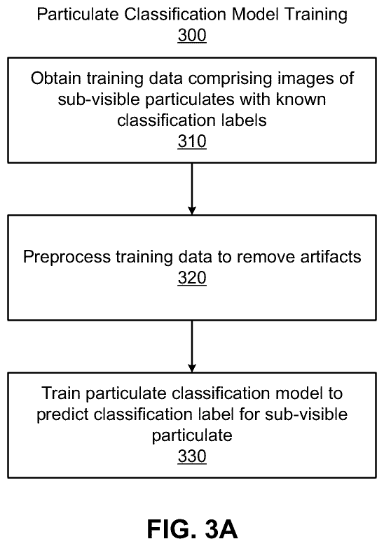

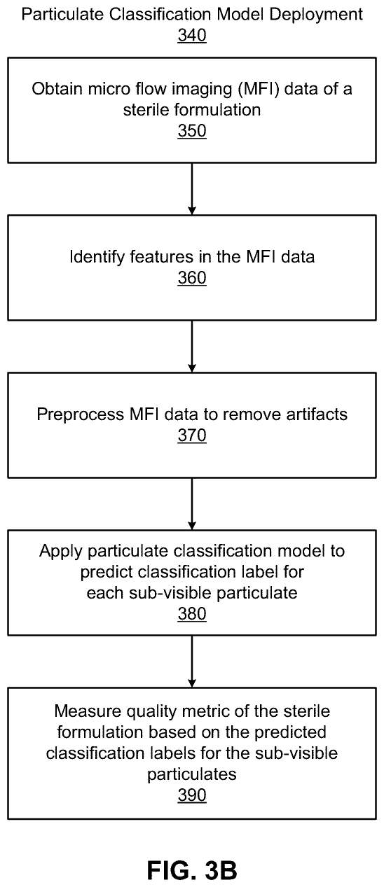

FIGS. 3A & 3B illustrate exemplary methods related to sub-visible particulate classification. In particular, the exemplary methods illustrated and described are described as being performed by the analysis system 130. However, some or all of the steps may be performed by other entities or components. In addition, some embodiments may perform the steps in parallel, perform the steps in different orders, or perform different steps.

FIG. 3A illustrates the method 300 of particulate classification model training, according to one or more embodiments.

The analysis system 130 obtains 310 training data comprising images of sub-visible particulates with known classification labels. Each image of a sub-visible particulate may be accompanied with a known classification label, e.g., annotated by a human expert. In other embodiments, the sub-visible particulate is unlabeled. The analysis system 130 may obtain the cropped images of the sub-visible particulates from MFI data, e.g., captured by the MFI system 120.

The analysis system 130 may preprocess 320 the training data to remove artifacts. In some embodiments, the analysis system 130 may identify features in the MFI data and distinguish whether the features are sub-visible particulates or artifacts. The analysis system may identify stripe-type artifacts and dirty-lens-type artifacts. Such artifacts may be excluded, while the non-artifact features are retained as sub-visible particulates. Other preprocessing steps include additional cropping of the sub-visible particulate images, obscuring the signal in the sub-visible particulate images (e.g., adding noise, rotating the images, removing portions of the image, etc.), other modification steps to prepare the training data.

The analysis system 130 trains 330 the particulate classification model to predict a classification label for a sub-visible particulate. The analysis system 130 may train the particulate classification model as a supervised machine-learning model (e.g., a CNN). In such embodiments, the analysis system 130 inputs the training data (i.e., the images of the sub-visible particulates) into the particulate classification model to output a prediction of the classification label. The analysis system 130 adjusts parameters of the particulate classification model to minimize a loss between the predictions of the model and the known classification labels of the training data. In other embodiments, the analysis system 130 may train the particulate classification model in an unsupervised manner, i.e., permitting the model to learn patterns within the training data without known labels enforced in the training.

Training of the particulate classification model may be performed through iterative batch training. Iterative batch training entails iteratively updating the model's parameters with batches of training samples. It strikes a balance between computational efficiency and convergence speed. The batch size is a hyperparameter that regulates model learning speed, the quality of the learning, and overall performance.

The trained particulate classification model is configured to input an image of a sub-visible particulate, e.g., derived from MFI data, and to output a predicted classification label of the sub-visible particulate. The trained particulate classification model may further output a confidence metric (e.g., high confidence, medium confidence, low confidence, or some other gradation) or a confidence score (e.g., confidence rated from 1 to 10, etc.) related to the prediction of the classification label. The confidence metric and/or score provides greater interpretability to the classification results, e.g., to a researcher or quality control technician.

FIG. 3B illustrates the method 340 of particulate classification model deployment, according to one or more embodiments.

The analysis system 130 obtaining 350 MFI data of a sterile formulation drug product. For example, the analysis system 130 may trigger generation of MFI data by the MFI system 120 (i.e., a pull configuration) or receive data on its generation (i.e., a push configuration). The MFI data includes one or more MFI samples, i.e., images, that depict subvisible particles in the sterile formulation drug product.

The analysis system 130 identifies 360 features in the MFI data. The features may be identified through a feature detection algorithm. For example, the algorithm may identify pixels that sufficiently contrast to a background noise signal. Each feature may be defined by a bounding box inclusive of pixels relating to the feature.

The analysis system 130 preprocesses 370 the MFI data to remove artifacts (e.g., stripe-type and dirty-lens-type artifacts). The stripe-type artifacts may be identified as having a bounding box below a certain aspect ratio, e.g., appearing as long boxes in the MFI data. The dirty-lens-type artifacts may be identified as features appearing in the same position in numerous micro flow images and, optionally, with a complex shape, indicating some contaminant on the lens of the MFI system. In some embodiments, the analysis system 130 performs the preprocessing to distinguish features as either sub-visible particulates or artifacts. Any feature that isn't detected as an artifact may be deemed a sub-visible particulate.

The analysis system 130 applies 380 the particulate classification model to predict a particulate classification label for each sub-visible particulate. The particulate classification model may input each cropped image of a sub-visible particulate to output a predicted classification label. In some embodiments, the particulate classification model may further output a confidence metric or confidence score related to the prediction for the sub-visible particulate. In some embodiments, the particulate classification model may flag low-confident predictions, i.e., indeterminate particulates, that may be presented to a user (e.g., a researcher or quality control technician) for human review. The label generated from the human review process may be used to retrain the classifier periodically to improve the future accuracy of the classifier.

The analysis system 130 measures 390 a quality metric of the sterile formulation based on the predicted classification labels for the sub-visible particulates. In some embodiments, the quality metric is based on a count of one or more particulate classification labels (e.g., labels pertaining to contaminants or undesirable particulates). In other embodiments, the quality metric is based on counts of each particulate classification label.

The analysis system 130 may return the quality metric and/or the predicted classification labels to a client device (e.g., the client device 140) for review by a user. For example, the analysis system 130 may prepare a report indicating the quality metric and/or providing the results of the particulate classification of the sub-visible particulates. The report may provide an interface for reviewing the particulate classification label and/or the confidence metric/score for each sub-visible particulate.

### Computing System Architecture

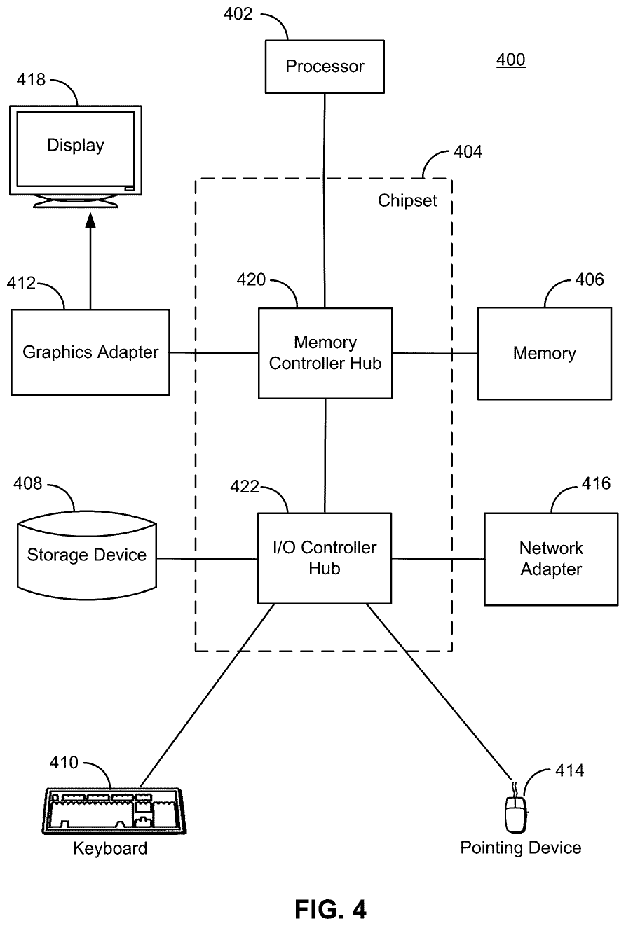

FIG. 4 is a block diagram of an example computer 400 suitable for use in the networked computing environment 100. The example computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, keyboard 410, pointing device 414, and network adapter 416 are coupled to the I/O controller hub 422. Other embodiments of the computer 400 have different architectures.

In the embodiment shown in FIG. 4, the storage device 408 is a non-transitory computer-readable storage medium such as a hard drive, compact disk read-only memory (CD-ROM), DVD, or a solid-state memory device. The memory 406 holds instructions and data used by the processor 402. The pointing device 414 is a mouse, track ball, touchscreen, or other type of pointing device, and may be used in combination with the keyboard 410 (which may be an on-screen keyboard) to input data into the computer system 400. The graphics adapter 412 displays images and other information on the display 418. The network adapter 416 couples the computer system 400 to one or more computer networks, such as network 150.

The types of computers used by the entities of FIGS. 1 and 2 can vary depending upon the embodiment and the processing power required by the entity. For example, the analysis system 130 might include multiple blade servers working together to provide the functionality described while a client device 140 might be a tablet or laptop. Furthermore, the computers can lack some of the components described above, such as keyboards 410, graphics adapters 412, and displays 418.

### Practical Applications

There are numerous applications of the particulate classification model. These various applications can improve drug manufacturing pipelines, thereby serving as technical improvements to the field.

In one application, the particulate classification methodology may be used to quality control drug formulation batches. Between manufacturing batches, the particulate classification model may be applied to samples to assess consistency in manufacturing. If a decline is identified in the quality, the analysis system may transmit a signal or notification to a user's client device to troubleshoot further. In additional embodiments, the particulate classification methodology may be used at various stages in the manufacturing process to individually assess each stage of the manufacturing process. This would provide insight into where quality degradation may occur. For example, if the quality of the drug formulation falters between two stages in the manufacturing process, the analysis system may determine and notify a source of the quality degradation. The methodology may also be used to assess differential manufacturing techniques and approaches. For example, a first manufacturing technique yields a formulation with a higher quality than a second manufacturing technique.

In another application, the particulate classification methodology may be used to characterize a drug formulation. Samples of the drug formulation may be subjected to varying conditions and tested to determine the effects of those conditions on quality of the drug formulation. For example, samples may be stored at different temperatures for a period of time and assessed for quality. The quality of each differentially-stored sample could provide insight into the shelf life of the drug formulation.

In yet another application, the particulate classification methodology may be used to compare different drug formulations. For example, a first drug formulation with one type of monoclonal antibody and a second drug formulation with a different type of monoclonal antibody are manufactured, both for treating a particular disease or illness. The particulate classification methodology may be used to assess quality of each formulation to determine an optimal candidate to produce and market.

### Example Results

The following figures and description relate to example results demonstrating the predictive power of the particulate classification model, according to one or more example implementations.

FIG. 5 illustrates a micro flow image 510, e.g., that may be captured by an MFI system. The micro flow image 510 is a two-dimensional image broken into coordinate axes. The MFI system and/or the analysis system may identify features in the micro flow image 510 with a bounding box encompassing each feature. The analysis system may perform preprocessing to distinguish between artifacts and particulates to be classified. Here, the analysis system identifies the lengthy bounding boxes as pertaining to stripe-type artifacts. Edge artifacts are also identified through the preprocessing.

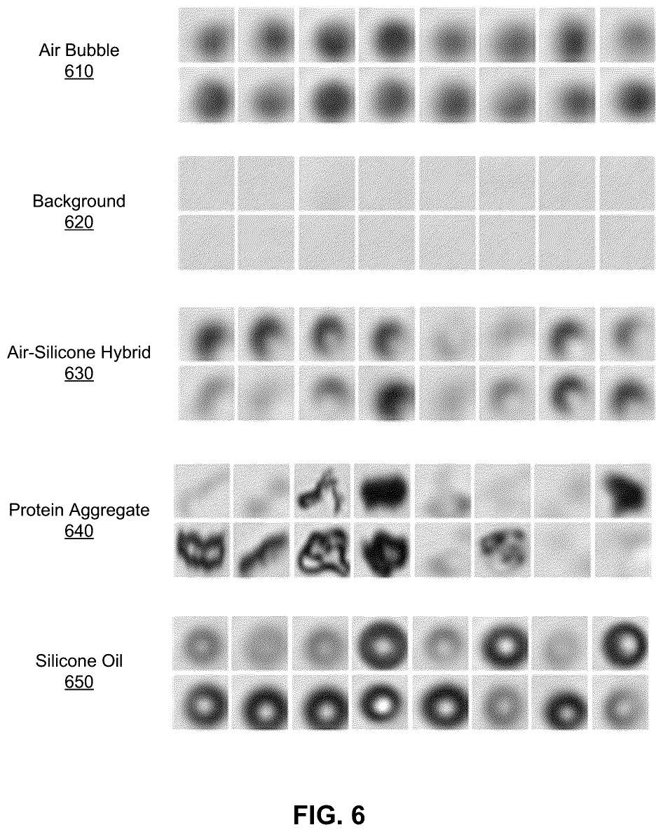

FIG. 6 illustrates example particulates classified into each classification label. In this example, the particulate classification model is trained to classify between five labels: Air Bubble 610, Background 620, Air-Silicone Hybrid 630, Protein Aggregate 640, and Silicone Oil 650. There's clear similarities between particulates within each class, demonstrating the predictive accuracy.

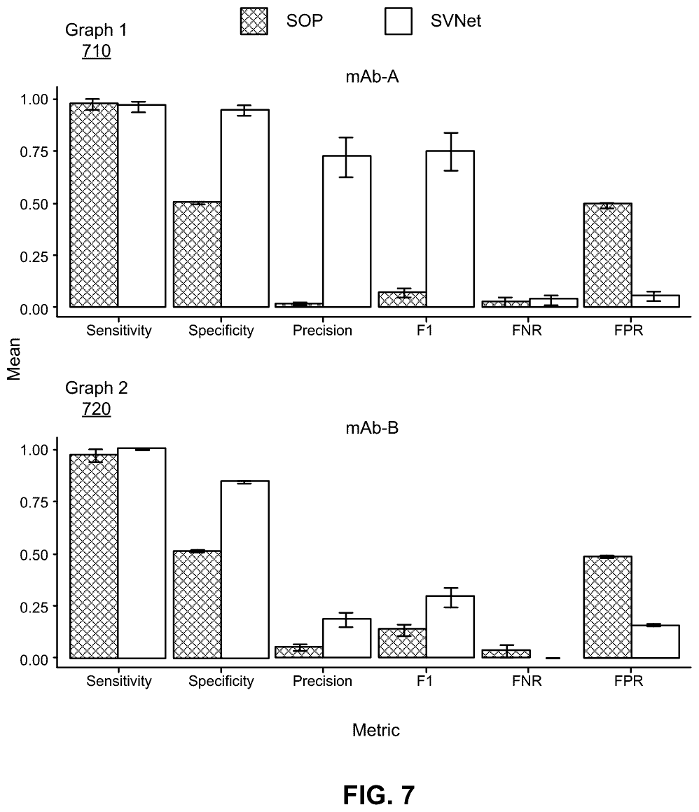

FIG. 7 illustrates example results comparing the particulate classification methodology with a conventional particulate screening methodology, according to an example implementation. The conventional methodology implements a rigid rule-based methodology that distinguishes protein aggregates versus air bubbles or silicone oil based on the sub-visible particulate's aspect ratio being above or below 0.85. If the aspect ratio is below 0.85, then the sub-visible particulate is deemed a protein aggregate, and otherwise deemed an air bubble or silicone oil. The convention methodology is referred to as “SOP” in the figure. The particulate classification model is referred to as “SVNet” in the figure. In Graph 1 710, the two methodologies are screening for the monoclonal antibody mAb-A, whereas Graph 2 720, the two methodologies screened for the monoclonal antibody mAb-B. The performance of the two methodologies are evaluated for sensitivity, specificity, precision, F-1 score (the harmonic mean of sensitivity and precision, false negative rate (FNR), and false positive rate (FPR). Notably, SVNet outperforms SOP in identifying both monoclonal antibodies, with better sensitivity, specificity, precision, and F-1 score. Also, notably, the false negative rate for SVNet was at (for mAb-A) or better (for mAb-B) than SOP, with the false positive rate greatly improved for both monoclonal antibodies.

### Additional Considerations

Some portions of above description describe the embodiments in terms of algorithmic processes or operations. These algorithmic descriptions and representations are commonly used by those skilled in the computing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs for a system and a process for automatically classifying subvisible particles. Thus, while particular embodiments and applications have been illustrated and described, it is to be understood that the described subject matter is not limited to the precise construction and components disclosed. The scope of protection should be limited only by any claims that may ultimately issue.

What is claimed is:

1. A computer-implemented method of sub-visible particle classification, the method comprising:
obtaining imaging data for a sterile formulation drug product, wherein the imaging data includes an image depicting a plurality of sub-visible particulates in the sterile formulation drug product;
detecting a plurality of features in the image, wherein each feature is bounded by a bounding box;
determining one or more of features to be artifacts based on characteristics of the features;
removing the artifacts from the plurality of features to generate a set of sub-visible particulates;
applying a particulate classification model to each sub-visible particulate to determine a particulate classification label, wherein the particulate classification model is a convolutional neural network; and
determining a quality metric for the sterile formulation drug product based on a count of sub-visible particulates in each particulate classification label.

2. The computer-implemented method of claim 1, wherein detecting the plurality of features comprises:
determining a background signal in the image covering a majority of pixels in the image; and
detecting features as pixels having a contrast to the background signal.

3. The computer-implemented method of claim 1, wherein detecting the plurality of features further comprises:
aggregating adjacent features into a single feature based on a distance of the two features being below a threshold distance.

4. The computer-implemented method of claim 1, wherein determining the one or more of features to be artifacts comprises:
determining an aspect ratio of the bounding box of each feature; and
determining a first feature to be a stripe-type artifact with the aspect ratio being below a threshold aspect ratio.

5. The computer-implemented method of claim 1, wherein determining the one or more features to be artifacts comprises:
determining a position of each feature in the image; and
determining a first feature to be a dirty-lens-type artifact with the position matching a position of a prior feature in a prior image in the imaging data.

6. The computer-implemented method of claim 1, wherein the particulate classification labels include: air bubble, silicone oil, air-silicone hybrid, protein aggregate, and background.

7. The computer-implemented method of claim 1, wherein the particulate classification model is trained with training data comprising images of sub-visible particulates annotated with particulate classification labels.

8. The computer-implemented method of claim 1, wherein the quality metric is based on a weight vector that scales each count of sub-visible particulates in each particulate classification label.

9. The computer-implemented method of claim 1, wherein the particulate classification model is further configured to output a confidence score for each sub-visible particulate.

10. The computer-implemented method of claim 1, further comprising:
returning the quality metric to a client device of a user and the counts of sub-visible particulates in the particulate classification labels.

11. A non-transitory computer-readable storage medium storing instructions for sub-visible particle classification, the instructions that, when executed by a computer processor, cause the computer processor to perform operations comprising:
obtaining imaging data for a sterile formulation drug product, wherein the imaging data includes an image depicting a plurality of sub-visible particulates in the sterile formulation drug product;
detecting a plurality of features in the image, wherein each feature is bounded by a bounding box;
determining one or more of features to be artifacts based on characteristics of the features;
removing the artifacts from the plurality of features to generate a set of sub-visible particulates;
applying a particulate classification model to each sub-visible particulate to determine a particulate classification label, wherein the particulate classification model is a convolutional neural network; and
determining a quality metric for the sterile formulation drug product based on a count of sub-visible particulates in each particulate classification label.

12. The non-transitory computer-readable storage medium of claim 11, wherein detecting the plurality of features comprises:
determining a background signal in the image covering a majority of pixels in the image; and
detecting features as pixels having a contrast to the background signal.

13. The non-transitory computer-readable storage medium of claim 11, wherein detecting the plurality of features further comprises:
aggregating adjacent features into a single feature based on a distance of the two features being below a threshold distance.

14. The non-transitory computer-readable storage medium of claim 11, wherein determining the one or more of features to be artifacts comprises:
determining an aspect ratio of the bounding box of each feature; and
determining a first feature to be a stripe-type artifact with the aspect ratio being below a threshold aspect ratio.

15. The non-transitory computer-readable storage medium of claim 11, wherein determining the one or more features to be artifacts comprises:
determining a position of each feature in the image; and
determining a first feature to be a dirty-lens-type artifact with the position matching a position of a prior feature in a prior image in the imaging data.

16. The non-transitory computer-readable storage medium of claim 11, wherein the particulate classification labels include: air bubble, silicone oil, air-silicone hybrid, protein aggregate, and background.

17. The non-transitory computer-readable storage medium of claim 11, wherein the particulate classification model is trained with training data comprising images of sub-visible particulates annotated with particulate classification labels.

18. The non-transitory computer-readable storage medium of claim 11, wherein the quality metric is based on a weight vector that scales each count of sub-visible particulates in each particulate classification label.

19. The non-transitory computer-readable storage medium of claim 11, wherein the particulate classification model is further configured to output a confidence score for each sub-visible particulate.

20. The non-transitory computer-readable storage medium of claim 11, the operations further comprising:
returning the quality metric to a client device of a user and the counts of sub-visible particulates in the particulate classification labels.
